(19) United States
(12) Patent Application Publication
(10) Pub. No.: US 2024/0012682 A1
(43) Pub. Date: Jan. 11, 2024

(54) PLACEMENT SEARCH DEVICE, PLACEMENT SEARCH METHOD AND PLACEMENT SEARCH PROGRAM

(71) Applicant: NIPPON TELEGRAPH AND TELEPHONE CORPORATION, Tokyo (JP)

(72) Inventors: Atsuhiko MAEDA, Tokyo (JP); Kenichi FUKUDA, Tokyo (JP); Yukio KIKUYA, Tokyo (JP); Sun Yeong KIM, Tokyo (JP); Kazuaki OBANA, Tokyo (JP)

(73) Assignee: NIPPON TELEGRAPH AND TELEPHONE CORPORATION, Tokyo (JP)

(21) Appl. No.: 18/033,971

(22) PCT Filed: Oct. 28, 2020
(86) PCT No.: PCT/JP2020/040462

§ 371 Date: Apr. 26, 2023

(51) Int. Cl. G06F 9/50; G06F 9/54

(52) U.S. Cl. CPC G06F 9/5005; G06F 9/542

(57) ABSTRACT

An allocation search device includes: a first sample output unit that outputs, on the basis of past event occurrence data, a first main sample that may occur under a predetermined condition and a first auxiliary sample that may occur under a similar condition; an allocation plan creation unit that creates a plurality of allocation plans of resources for an occurring event; a first allocation evaluation unit that evaluates whether or not each of the plurality of allocation plans satisfies a predetermined evaluation criterion in a case where the first main sample and the first auxiliary sample are applied to each of the plurality of allocation plans; a second sample output unit that outputs, on the basis of latest event occurrence data, a future second main sample that may occur under the predetermined condition and a future second auxiliary sample that may occur under the similar condition; and a second allocation evaluation unit that, in a case where the second main sample and the second auxiliary sample are applied to each of the effective allocation plans that satisfy the predetermined evaluation criterion, reevaluates whether or not each of the effective allocation plans satisfies the predetermined evaluation criterion.

### TECHNICAL FIELD

The disclosed technology relates to an allocation search device, allocation search method, and allocation search program.

### BACKGROUND ART

There is known a technology of predicting occurrence of an event in each region and optimally allocating a plurality of resources for the event. For example, Non Patent Literature 1 proposes a method of predicting a regional emergency demand and optimally allocating a plurality of ambulance squads (ambulances) on the basis of the prediction so that a time required to arrive at a site or a travel distance required to arrive at a site is reduced as much as possible.

### CITATION LIST

### Non Patent Literature

Non Patent Literature 1: “Confirmation of effectiveness of emergency vehicle optimal operation system using emergency big data—aiming to reduce ambulance transport time on the basis of real-time emergency demand prediction—” https://www.ntt.co.jp/news2018/1811/181126a.html

### SUMMARY OF INVENTION

### Technical Problem

For example, there is a case where occurrence of a sick/injured person per unit time is predicted in each region mesh of 500 m square or 1 km square, and a plurality of ambulance squads (ambulances) is appropriately allocated so that a time required to arrive at a site or a travel distance required to arrive at a site is reduced as much as possible. The ambulance squads can be allocated in a plurality of fire stations existing in a target region, and one ambulance squad waiting at the shortest distance from a site of occurrence of a sick/injured person is dispatched. In this case, the plurality of ambulance squads is to be appropriately allocated in the plurality of fire stations. Thus, a discrete optimization problem arises.

However, for example, there are about 50 fire stations and about 40 ambulance squads in core cities in Japan. In this case, for example, when it is simplified that any number of ambulance squads can be allocated in each fire station, the number of allocation patterns is 50 to the 40th power by simple calculation. Therefore, it is difficult to find an optimal solution of such a problem in real time.

Meanwhile, even if occurrence of a sick/injured person is predicted in each regional mesh, a result thereof may be wrong. Regarding this point, it is desirable that ambulance squads be in robust allocation that can expect an effect even if the prediction is not completely right.

The disclosed technology has been made in view of the above points, and an object thereof is to provide an allocation search device, allocation search method, and allocation search program capable of obtaining, in a short time during which it is necessary to determine effective allocation of resources, robust allocation of the resources that can expect an effect even if a prediction result of occurrence of an event is not completely right.

### Solution to Problem

In order to achieve the above object, an allocation search device according to an aspect of the present disclosure includes: a first sample output unit configured to output, on the basis of event occurrence data obtained in the past, event occurrence data that may occur under a predetermined condition as a first main sample and event occurrence data that may occur under a condition similar to the predetermined condition as a first auxiliary sample; an allocation plan creation unit configured to create a plurality of allocation plans of resources for an occurring event; a first allocation evaluation unit configured to evaluate whether or not each of the plurality of allocation plans satisfies a predetermined evaluation criterion in a case where the first main sample and the first auxiliary sample are applied to each of the plurality of allocation plans; a second sample output unit configured to output, on the basis of latest event occurrence data, future event occurrence data that may occur under the predetermined condition as a second main sample and future event occurrence data that may occur under the similar condition as a second auxiliary sample; and a second allocation evaluation unit configured to, in a case where the second main sample and the second auxiliary sample are applied to each of the allocation plans that satisfy the predetermined evaluation criterion, reevaluate whether or not each of the allocation plans that satisfy the predetermined evaluation criterion satisfies the predetermined evaluation criterion.

In order to achieve the above object, an allocation search method according to an aspect of the present disclosure includes: outputting, on the basis of event occurrence data obtained in the past, event occurrence data that may occur under a predetermined condition as a first main sample and event occurrence data that may occur under a condition similar to the predetermined condition as a first auxiliary sample; creating a plurality of allocation plans of resources for an occurring event; evaluating whether or not each of the plurality of allocation plans satisfies a predetermined evaluation criterion in a case where the first main sample and the first auxiliary sample are applied to each of the plurality of allocation plans; outputting, on the basis of latest event occurrence data, future event occurrence data that may occur under the predetermined condition as a second main sample and future event occurrence data that may occur under the similar condition as a second auxiliary sample; and, in a case where the second main sample and the second auxiliary sample are applied to each of the allocation plans that satisfy the predetermined evaluation criterion, reevaluating whether or not each of the allocation plans that satisfy the predetermined evaluation criterion satisfies the predetermined evaluation criterion.

In order to achieve the above object, an allocation search program according to an aspect of the present disclosure causes a computer to execute: outputting, on the basis of event occurrence data obtained in the past, event occurrence data that may occur under a predetermined condition as a first main sample and event occurrence data that may occur under a condition similar to the predetermined condition as a first auxiliary sample; creating a plurality of allocation plans of resources for an occurring event; evaluating whether or not each of the plurality of allocation plans satisfies a predetermined evaluation criterion in a case where the first main sample and the first auxiliary sample are applied to each of the plurality of allocation plans; outputting, on the basis of latest event occurrence data, future event occurrence data that may occur under the predetermined condition as a second main sample and future event occurrence data that may occur under the similar condition as a second auxiliary sample; and, in a case where the second main sample and the second auxiliary sample are applied to each of the allocation plans that satisfy the predetermined evaluation criterion, reevaluating whether or not each of the allocation plans that satisfy the predetermined evaluation criterion satisfies the predetermined evaluation criterion.

### Advantageous Effects of Invention

According to the disclosed technology, it is possible to obtain, in a short time during which it is necessary to determine effective allocation of resources, robust allocation of the resources that can expect an effect even if a prediction result of occurrence of an event is not completely right.

### BRIEF DESCRIPTION OF DRAWINGS

FIG. 1 is a block diagram illustrating an example of a hardware configuration of an allocation search device according to an embodiment.

FIG. 2 is a block diagram illustrating an example of functional configurations of an allocation search device according to an embodiment.

FIG. 3 shows an example of sick/injured person occurrence data rows according to an embodiment.

FIG. 4 shows an example of pseudo occurrence data rows according to an embodiment.

FIG. 5 shows another example of pseudo occurrence data rows according to an embodiment.

FIG. 6 is graphs showing an example of a sick/injured person occurrence probability obtained by MCMC according to an embodiment.

FIG. 7 shows an example of ambulance data according to an embodiment.

FIG. 8 shows an example of fire station data according to an embodiment.

FIG. 9 shows an example of ambulance allocation plans according to an embodiment.

FIG. 10 shows an example of original ambulance allocation according to an embodiment.

FIG. 11 shows an example of effective allocation plans that satisfy evaluation criterion according to an embodiment.

FIG. 12 shows an example of allocation plans that do not satisfy evaluation criterion according to an embodiment.

FIG. 13 is a flowchart showing an example of a flow of processing in phase 1 by an allocation search program according to an embodiment.

FIG. 14 is a flowchart showing an example of a flow of evaluation processing according to an embodiment.

FIG. 15 is a flowchart showing an example of a flow of processing in phase 2 by an allocation search program according to an embodiment.

### DESCRIPTION OF EMBODIMENTS

Hereinafter, an example of an embodiment of the disclosed technology will be described with reference to the drawings. In the drawings, the same or equivalent components and portions will be denoted by the same reference signs. Further, dimensional ratios in the drawings are exaggerated for convenience of description and thus may be different from actual ratios.

In this embodiment, there will be described an aspect in which a regional emergency demand is predicted and a plurality of ambulance squads (ambulances) is optimally allocated on the basis of the prediction so that a time required to arrive at a site or a travel distance required to arrive at a site is reduced as much as possible. However, this embodiment can be applied as long as resources can be optimally allocated for an occurring event.

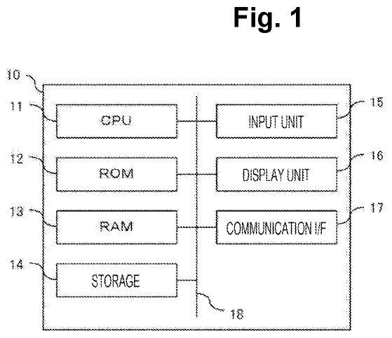

FIG. 1 is a block diagram illustrating an example of a hardware configuration of an allocation search device 10 according to this embodiment.

As illustrated in FIG. 1, the allocation search device 10 includes a central processing unit (CPU) 11, a read only memory (ROM) 12, a random access memory (RAM) 13, a storage 14, an input unit 15, a display unit 16, and a communication interface (I/F) 17. The components are communicably connected to each other via a bus 18.

The CPU 11 is a central processing unit, and executes various programs and controls each unit. That is, the CPU 11 reads the programs from the ROM 12 or the storage 14 and executes the programs by using the RAM 13 as a work area. The CPU 11 controls each component described above and performs various types of operation processing according to the programs stored in the ROM 12 or the storage 14. In this embodiment, the ROM 12 or the storage 14 stores an allocation search program for searching for optimal allocation of resources.

The ROM 12 stores various programs and various types of data. The RAM 13 temporarily stores the programs or data as a work area. The storage 14 includes a hard disk drive (HDD) or a solid state drive (SSD) and stores various programs including an operating system and various types of data.

The input unit 15 includes a pointing device such as a mouse and a keyboard and is used to perform various inputs to the allocation search device.

The display unit 16 is, for example, a liquid crystal display and displays various types of information. The display unit 16 may function as the input unit 15 by adopting a touchscreen system.

The communication interface 17 is an interface through which the allocation search device communicates with another external device. The communication is performed in conformity to, for example, a wired communication standard such as Ethernet (registered trademark) or fiber distributed data interface (FDDI) or a wireless communication standard such as 4G, 5G, or Wi-Fi (registered trademark).

For example, a general-purpose computer device such as a server computer or personal computer (PC) is applied to the allocation search device 10 according to this embodiment.

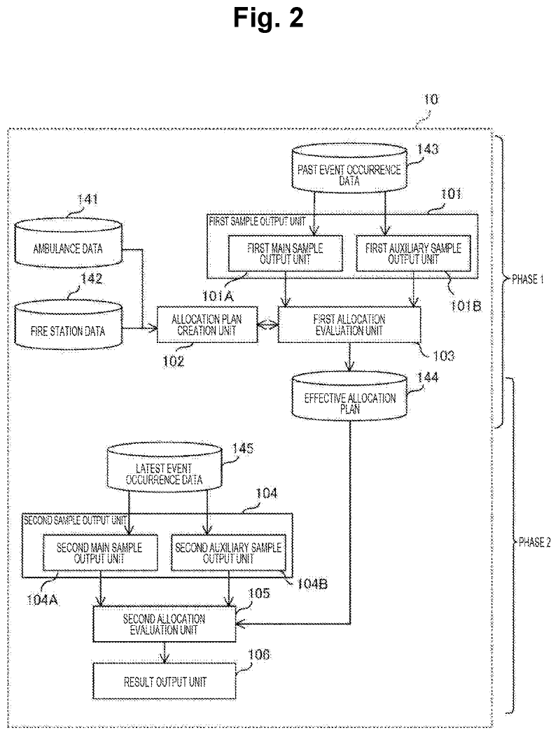

Next, functional configurations of the allocation search device 10 will be described with reference to FIG. 2.

FIG. 2 is a block diagram illustrating an example of the functional configurations of the allocation search device 10 according to this embodiment.

As illustrated in FIG. 2, the allocation search device 10 includes, as the functional configurations, a first sample output unit 101, an allocation plan creation unit 102, a first allocation evaluation unit 103, a second sample output unit 104, a second allocation evaluation unit 105, and a result output unit 106. Each functional configuration is achieved by the CPU 11 reading the allocation search program stored in the ROM 12 or the storage 14, developing the allocation search program in the RAM 13, and executing the allocation search program.

The first sample output unit 101 includes a first main sample output unit 101A and a first auxiliary sample output unit 101B, and the second sample output unit 104 includes a second main sample output unit 104A and a second auxiliary sample output unit 104B.

The storage 14 stores ambulance data 141, fire station data 142, past event occurrence data 143, an effective allocation plan 144, and latest event occurrence data 145. The ambulance data 141, the fire station data 142, the past event occurrence data 143, the effective allocation plan 144, and the latest event occurrence data 145 may be stored in an external storage device.

The past event occurrence data 143 is a data row of event occurrence data obtained in the past. The past herein means a certain period in the past from a current point of time at which allocation search is performed and is, for example, a period of past several months or past several years. The latest event occurrence data 145 is a data row of the latest event occurrence data. The latest herein means a certain period immediately before a current point of time at which allocation search is performed and is, for example, a period of the last several days or the last several months. That is, the latest period is shorter than the past period. The event occurrence data is, for example, sick/injured person occurrence data (i.e., data indicating the year, month, date, day, hour, minute, longitude, and latitude at which a sick/injured person occurs).

Based on the past event occurrence data 143, the first sample output unit 101 outputs event occurrence data that may occur under a predetermined condition as a first main sample and outputs event occurrence data that may occur under a condition similar to the predetermined condition as a first auxiliary sample. The predetermined condition is, for example, a condition such as 10:00 on weekdays in September, and the condition similar to the condition is, for example, a condition such as 10:00 on weekdays in August and October. In this embodiment, the first main sample output unit 101A outputs the first main sample, and the first auxiliary sample output unit 101B outputs the first auxiliary sample.

The first main sample may be expressed as, for example, a pseudo occurrence data row generated in a pseudo manner in accordance with an event occurrence frequency that is obtained in each certain area on the basis of a data row that actually occurs under the predetermined condition. Similarly, the first auxiliary sample may be expressed as, for example, a pseudo occurrence data row generated in a pseudo manner in accordance with an event occurrence frequency that is obtained in each certain area on the basis of a data row that actually occurs under the condition similar to the predetermined condition. The first main sample may also be expressed as a pseudo occurrence data row generated in a pseudo manner in accordance with a random variable of an event occurrence probability that is obtained on the basis of the data row that actually occurs under the predetermined condition. Similarly, the first auxiliary sample may also be expressed as a pseudo occurrence data row generated in a pseudo manner in accordance with a random variable of an event occurrence probability that is obtained on the basis of the data row that actually occurs under the condition similar to the predetermined condition. Specific examples of the event occurrence frequency and the event occurrence probability will be described later.

The allocation plan creation unit 102 creates a plurality of allocation plans of resources for an occurring event. In this embodiment, the allocation plan creation unit creates a plurality of allocation plans of ambulances for allocating the ambulances to fire stations by using the ambulance data 141 and the fire station data 142.

The first allocation evaluation unit 103 uses the first main sample, the first auxiliary sample, and the plurality of allocation plans as inputs and evaluates whether or not each of the plurality of allocation plans satisfies a predetermined evaluation criterion in a case where the first main sample and the first auxiliary sample are applied to each of the plurality of allocation plans. The first allocation evaluation unit 103 stores an allocation plan that satisfies the predetermined evaluation criterion in the storage 14 as the effective allocation plan 144. The above processing is “phase 1”.

Next, based on the latest event occurrence data 145, the second sample output unit 104 outputs future event occurrence data that may occur under a predetermined condition as a second main sample and outputs future event occurrence data that may occur under a condition similar to the predetermined condition as a second auxiliary sample. Both the predetermined condition and the condition similar to the predetermined condition are the same as the conditions in the first sample output unit 101. In this embodiment, the second main sample output unit 104A outputs the second main sample, and the second auxiliary sample output unit 104B outputs the second auxiliary sample.

The second allocation evaluation unit 105 uses the second main sample, the second auxiliary sample, and the effective allocation plan 144 as inputs and reevaluates whether or not each effective allocation plan 144 satisfies a predetermined evaluation criterion in a case where the second main sample and the second auxiliary sample are applied to each effective allocation plan 144.

As a result of the reevaluation by the second allocation evaluation unit 105, the result output unit 106 outputs an effective allocation plan that satisfies the predetermined evaluation criterion as optimal allocation of the resources. The above processing is “phase 2”.

This embodiment roughly includes two implementation phases, i.e., phase 1 and phase 2, as described above. Phase 1 is a phase in which a large number of allocation patterns in effective ambulance allocation plans are found in advance on the basis of past sick/injured person occurrence data. Phase 1 is performed, for example, at the beginning of the year, every quarter, or once a month. Phase 2 is a phase in which, for example, occurrence of a sick/injured person in near future is predicted on the basis of the latest sick/injured person occurrence data at the same time every day, the most effective allocation pattern is found from the effective allocation patterns found in advance in the phase 1, and allocation is changed according to the most effective allocation pattern.

First, the processing in phase 1 will be described by exemplifying a case where, for example, optimal allocation of ambulances between 10:00 and 11:00 on weekdays in the next month September is obtained in August. Based on the past sick/injured person occurrence data accumulated by August, the first main sample output unit 101A outputs a plurality of occurrence data rows predicted to be most likely to occur under a predetermined condition (e.g. between 10:00 and 11:00 on weekdays in September) as the first main sample. Further, based on the past sick/injured person occurrence data accumulated by August, the first auxiliary sample output unit 101B outputs a plurality of occurrence data rows predicted to be likely to occur under a condition similar to the predetermined condition (e.g. between 10:00 and 11:00 on weekdays in August and October) as the first auxiliary sample.

There is a plurality of methods of outputting the first main sample and the first auxiliary sample. A first method, which is the simplest method, is to output actual occurrence data rows between 10:00 and 11:00 on weekdays in September for the past several years as they are as the first main sample and output actual occurrence data rows between 10:00 and 11:00 on weekdays in August and October for the past several years as the first auxiliary sample. The samples are processed as described above on the following two assumptions: similar sick/injured person occurrence patterns occur in the same month, the same day, and the same time slot every year; and the occurrence data rows in August and October are similar to the occurrence data rows in September because, for example, average daily temperatures in August and October are relatively close to that in September.

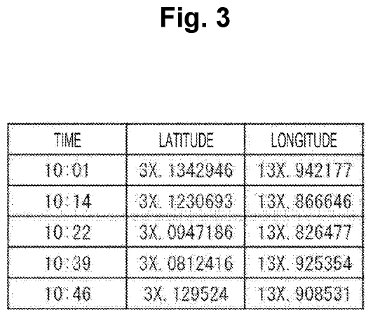

FIG. 3 shows an example of the sick/injured person occurrence data rows according to this embodiment.

The sick/injured person occurrence data rows in FIG. 3 are an example of occurrence data rows for a period of time between 10:00 and 11:00 on one weekday, which are output as the first main sample by the first main sample output unit 101A by the above method. In the example of FIG. 3, information regarding the year, month, date, and day is not output because the information is unnecessary for the subsequent processing. Data only for one day is output in the example of FIG. 3, but, in practice, data for a plurality of days (e.g. 100 days if the corresponding number of days is 100 days) is output. The same applies to the sick/injured person occurrence data rows output as the first auxiliary sample by the first auxiliary sample output unit 101B. In this method, the number of days output from the first auxiliary sample output unit 101B is generally larger than the number of days output from the first main sample output unit 101A.

As a second method, which is another simple method, actual occurrence data rows between 10:00 and 11:00 on weekdays in September for the past several years may be output as they are as the first main sample, and actual occurrence data rows between 10:00 and 11:00 on weekdays from January to December including a period of time between 10:00 and 11:00 on weekdays in September for the past several years may be output as the first auxiliary sample.

As a third method, which is still another method, a method of creating and using pseudo occurrence data rows will be described. Specifically, the first main sample output unit 101A obtains a sick/injured person occurrence frequency in a certain area (e.g. every 500 m square or every 1 km square) on the basis of the actual occurrence data rows between 10:00 and 11:00 on weekdays in September for the past several years and generates pseudo occurrence data rows in accordance with the frequency. The sick/injured person occurrence frequency is an example of the event occurrence frequency. For example, the sick/injured person occurrence frequency in a certain area of 500 m square between 10:00 and 11:00 on weekdays in September is obtained as 30/100=0.3, where the past occurrence data rows to be used are, for example, data for 100 days and the total number of occurrence of sick/injured people during the period is, for example, 30 people. Similarly, the occurrence frequency may be obtained in all areas of a target region, pseudo occurrence data rows corresponding to values of the occurrence frequencies may be generated, and the data rows in all the areas may be used as one set. Regarding in which position in the target area a sick/injured person occurs, a sick/injured person may occur on the basis of, for example, a density obtained by kernel density estimation that is performed by plotting past actual occurrence positions. An advantage of the method of creating pseudo occurrence data rows is that robust verification using more occurrence patterns can be performed by creating more samples than occurrence data rows that have actually occurred in the past.

At this time, the auxiliary sample output unit 101B may increase or decrease 0.3 that is a value of the occurrence frequency calculated as described above by a certain value (e.g. increase or decrease 0.3 by 0.05 every time, thereby obtaining values of 0.35 and 0.25) and generate pseudo occurrence data rows on the basis of the values. Alternatively, as in the above example, the auxiliary sample output unit may obtain the occurrence frequency on the basis of the past occurrence data rows in August and October and then generate pseudo occurrence data rows.

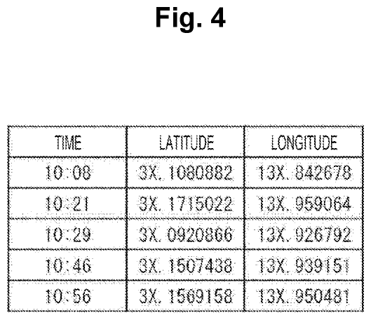

FIG. 4 shows an example of the pseudo occurrence data rows according to this embodiment.

The pseudo occurrence data rows in FIG. 4 are obtained by obtaining an occurrence frequency in each area, generating pseudo occurrence data, and summarizing the pseudo occurrence data as one set of data rows. The occurrence frequency is the same as that in the above example of FIG. 3.

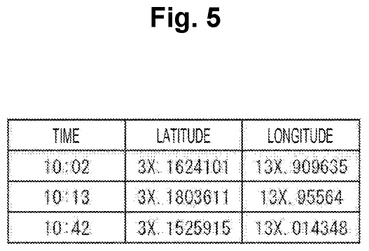

FIG. 5 shows another example of the pseudo occurrence data rows according to this embodiment.

The pseudo occurrence data rows in FIG. 5 are created after an occurrence frequency is obtained in each area and then the occurrence frequency in each area is decreased by a certain value. As a result, the total number of occurrences between 10:00 and 11:00 is smaller than that in the example of FIG. 4.

As a fourth method, which is further another method, a method of obtaining a sick/injured person occurrence probability as a random variable and creating pseudo occurrence data will be described. The sick/injured person occurrence probability is an example of the event occurrence probability. Obtaining the sick/injured person occurrence probability as the random variable means that a possibility that the sick/injured person occurrence probability takes various values is considered, for example, a possibility that 0.3 people occur is 50%, a possibility that 0.31 people occur is 10%, a possibility that 0.32 people occur is 5%, . . . , and each possibility is expressed as a probability. In order to obtain the sick/injured person occurrence probability as the random variable, target past occurrence data rows may be assumed to occur according to the Poisson distribution, and a parameter of the Poisson distribution (indicating how many times a sick/injured person occurs within a certain period of time) may be obtained as the random variable by using the Markov chain Monte Carlo method (MCMC) or the like. From this result, the first main sample output unit 101A may generate pseudo occurrence data on the basis of a parameter having the highest probability, and the first auxiliary sample output unit 101B may generate pseudo occurrence data on the basis of parameters having other probabilities. In practice, the occurrence probability has a continuous distribution, and thus the pseudo occurrence data may be generated by picking up parameters at certain intervals from the distribution.

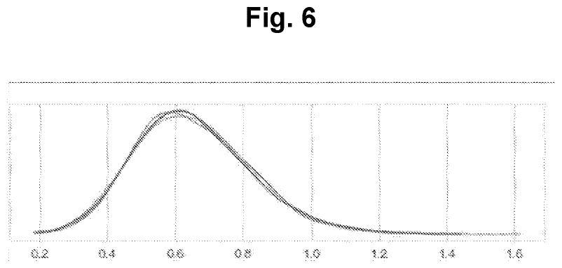

FIG. 6 is graphs showing an example of the sick/injured person occurrence probability obtained by MCMC according to this embodiment.

The graphs in FIG. 6 visualize a result of obtaining the parameter of the Poisson distribution as the random variable as described above. The horizontal axis represents a value of the parameter of the Poisson distribution, and the vertical axis represents a probability thereof. There are four graphs in the example of FIG. 6, and each graph corresponds to a chain of MCMC. This indicates that the graphs are calculated by using four chains. The four graphs are approximately overlapped. This indicates that the calculation of MCMC is approximately convergent. In this case, the first main sample output unit 101A generates occurrence data in a pseudo manner in accordance with the Poisson distribution of a parameter of 0.6 that is the highest probability. Meanwhile, the first auxiliary sample output unit 101B generates occurrence data on the basis of, for example, parameters 0.2, 0.4, 0.8, and 1.0 around 0.6.

In a case where both the first main sample output unit 101A and the first auxiliary sample output unit 101B generate pseudo occurrence data rows, the number of days for the first auxiliary sample output unit 101B may be intentionally reduced. However, the length of the data rows is not reduced. The length of the data rows increases as the parameter of the occurrence probability increases. In a case where the parameter of the Poisson distribution is obtained as the random variable by MCMC described above, the number of days of data generation may be determined according to the magnitude of the probability. In the example of FIG. 6 described above, the number of samples is reduced in the order of the parameters 0.6, 0.8, 0.4, 1.0, and 0.2. The first allocation evaluation unit 103 in the subsequent stage basically performs evaluation while putting a weight on the data rows output by the first main sample output unit 101A. However, in a case where the number of days of data rows output by the first auxiliary sample output unit 101B is intentionally reduced to be smaller than the number of days output by the first main sample output unit 101A, the first allocation evaluation unit performs equivalent processing even when the first allocation evaluation unit performs evaluation with a weighted average on the basis of a unified evaluation criterion.

The occurrence data rows created by the first main sample output unit 101A and the first auxiliary sample output unit 101B are output to the first allocation evaluation unit 103.

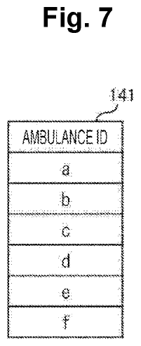

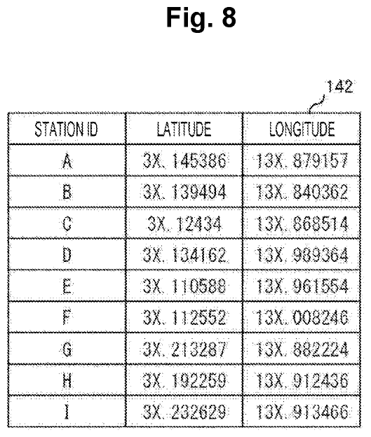

Meanwhile, the allocation plan creation unit 102 creates, for example, a plurality of allocation plans for allocating ambulances to fire stations on the basis of the ambulance data 141 in FIG. 7 and the fire station data 142 in FIG. 8.

FIG. 7 shows an example of the ambulance data 141 according to this embodiment. FIG. 8 shows an example of the fire station data 142 according to this embodiment.

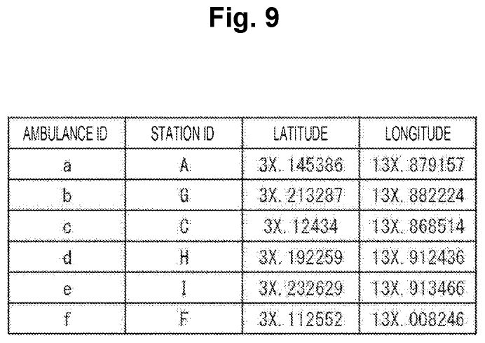

In the example of the ambulance data 141 in FIG. 7, six ambulances having ambulance identifications (IDs) a to f are registered. In the example of the fire station data 142 in FIG. 8, nine fire stations having fire station IDs A to I are registered. As a method of creating a first allocation plan, for example, allocation plans may be randomly created as shown in FIG. 9.

FIG. 9 shows an example of ambulance allocation plans according to this embodiment. Unique IDs (not illustrated) are given to the plurality of allocation plans created by the allocation plan creation unit 102.

The allocation plans created by the allocation plan creation unit 102 are output to the first allocation evaluation unit 103. There are various methods of creating the second and subsequent allocation plans. The simplest method is a method of also randomly creating the second and subsequent allocation plans. However, in this case, in a case where the number of combinations is enormous, a long time may be required until an allocation plan evaluated to be effective by the first allocation evaluation unit 103 is specified. In view of this, various heuristics (also referred to as heuristic methods) can be used. One of the methods is a method using a genetic algorithm.

An example of the method using the genetic algorithm will be described. An allocation plan is randomly created until an allocation plan evaluated to be effective by the first allocation evaluation unit 103 is specified, and, in a case where an allocation plan is evaluated to be effective, a next allocation plan is created by randomly changing a part of the allocation plan on the basis of the allocation plan or combining a plurality of allocation plans evaluated to be effective. Combining the plurality of allocation plans means that, for example, allocation of the ambulances a to c is extracted from one allocation plan, allocation of the ambulances d to f is extracted from another allocation plan, and the allocations are combined. In this way, it is empirically known that a solution close to an optimal solution can be obtained in a relatively short time.

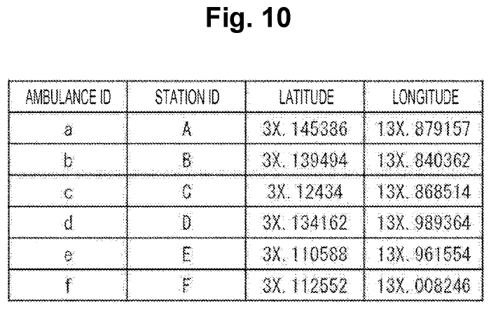

Next, the first allocation evaluation unit 103 evaluates the allocation plans acquired from the allocation plan creation unit 102 by using the occurrence data rows acquired from both the first main sample output unit 101A and the first auxiliary sample output unit 101B. As an evaluation method, for example, an average of distances that dispatchable ambulances existing closest to a site of occurrence of a sick/injured person travel until the ambulances arrive at the site is calculated, and the calculated average travel distance is compared with, for example, an average travel distance in original ambulance allocation in FIG. 10.

FIG. 10 shows an example of the original ambulance allocation according to this embodiment.

In a case where a certain ambulance is dispatched, the certain ambulance cannot respond to the next request for dispatch for a certain period of time. The certain period of time may be, for example, a value of an average time required to complete transport of a sick/injured person and obtained in advance. For example, in a case where the average time required to complete transport of a sick/injured person is 50 minutes, the certain ambulance cannot respond to the next request for dispatch for 50 minutes. As a method of obtaining a distance from a site of occurrence of a sick/injured person, for example, a direct distance may be used most simply, or the shortest distance on a road network may be used in a case where road network data can be prepared.

In a case where the first main sample output unit 101A outputs data for 100 days, for example, the first allocation evaluation unit 103 evaluates all the data for 100 days. The same applies to the data output from the first auxiliary sample output unit 101B.

As initial values of states of the ambulances, in practice, there is a possibility that several ambulances are already dispatched. Therefore, for example, it is desirable to perform evaluation in various initial states for each sample for one day. The initial states are, for example, a state in which the ambulance a is currently dispatched and returns in 30 minutes and a state in which the ambulances a and b are currently dispatched, the ambulance a can respond in 30 minutes, and the ambulance b can respond in 40 minutes.

As a result of the evaluation described above, in a case where an allocation plan satisfies the predetermined evaluation criterion, the allocation plan is regarded as effective and is stored in the storage 14 as the effective allocation plan 144.

At this time, basically, the occurrence data rows acquired from the first main sample output unit 101A and the occurrence data rows acquired from the first auxiliary sample output unit 101B have different importance and therefore may be evaluated on the basis of different evaluation criteria. For example, in the occurrence data rows acquired from the first main sample output unit 101A, the allocation plan satisfies the evaluation criterion in a case where an average distance required to arrive at a site (hereinafter, referred to as a “site arrival distance”) is shortened from an average distance in original allocation serving as a reference by 100 m or more on average. Meanwhile, in the occurrence data rows acquired from the first auxiliary sample output unit 101B, the allocation plan satisfies the evaluation criterion in a case where the average site arrival distance is shortened by 50 m or more.

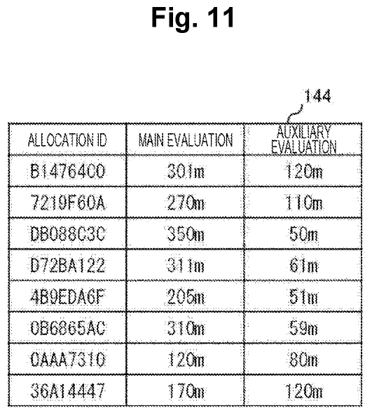

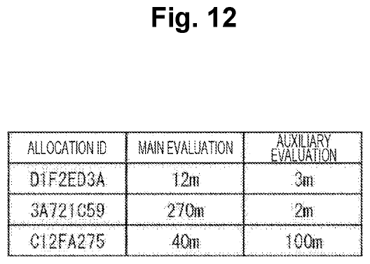

FIG. 11 shows an example of the effective allocation plans 144 that satisfy the evaluation criterion according to this embodiment. FIG. 12 shows an example of allocation plans that do not satisfy the evaluation criterion according to this embodiment.

In the effective allocation plans 144 in FIG. 11, a main evaluation and an auxiliary evaluation are associated with an allocation ID indicating an allocation plan. The main evaluation indicates a distance difference obtained in a case where the occurrence data rows acquired from the first main sample output unit 101A are applied to the allocation plan, and the auxiliary evaluation indicates a distance difference obtained in a case where the occurrence data rows acquired from the first auxiliary sample output unit 101B are applied to the allocation plan. The distance difference herein indicates, as described above, a difference between the average site arrival distance obtained in a case where the occurrence data rows are applied to the allocation plan and the average site arrival distance obtained in a case where the occurrence data rows are applied to the original allocation. The evaluation criteria are different criteria between the main evaluation and the auxiliary evaluation as described above (e.g. the shortened distance difference is 100 m or more on average in the main evaluation, and the shortened distance difference is 50 m or more on average in the auxiliary evaluation). That is, the effective allocation plans 144 in FIG. 11 satisfy the evaluation criteria in both the main evaluation and the auxiliary evaluation.

Meanwhile, the allocation plans in FIG. 12 do not satisfy the evaluation criteria. For example, an allocation plan having an allocation ID of D1F2ED3A does not satisfy the evaluation criteria in either the main evaluation or the auxiliary evaluation. An allocation plan having an allocation ID of 3A721C59 satisfies the evaluation criterion in the main evaluation, but does not satisfy the evaluation criterion in the auxiliary evaluation. On the contrary, an allocation plan having an allocation ID of C12FA275 does not satisfy the evaluation criterion in the main evaluation, but satisfies the evaluation criterion in the auxiliary evaluation.

In a case where the number of days in the occurrence data rows output by the first auxiliary sample output unit 101B is intentionally reduced to be smaller than the number of days output by the first main sample output unit 101A as described above, evaluation may be performed with a weighted average on the basis of a unified evaluation criterion.

The first allocation evaluation unit 103 may output an evaluation result of a certain allocation plan to the allocation plan creation unit 102 and reflect the evaluation result in creation of the next allocation plan.

As described above, it is possible to empirically find several tens to several hundreds of effective allocation patterns by repeating the above processing in phase 1 for more than ten hours by using a general-purpose PC or the like.

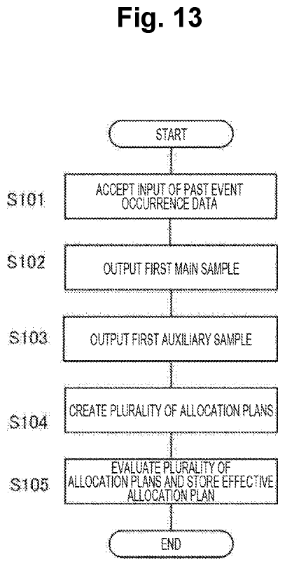

Next, an operation of the allocation search device 10 according to this embodiment will be described with reference to FIG. 13.

FIG. 13 is a flowchart showing an example of a flow of processing in phase 1 by an allocation search program according to this embodiment. The processing in phase 1 by the allocation search program is implemented by the CPU 11 of the allocation search device 10 writing the allocation search program stored in the ROM 12 or the storage 14 to the RAM 13 and executing the allocation search program.

In step S101 of FIG. 13, the CPU 11 accepts input of the past event occurrence data 143 indicating, for example, event occurrence data for the past several years. In this example, the event occurrence data indicates, for example, the sick/injured person occurrence data as described above.

In step S102, the CPU 11 outputs event occurrence data that may occur under a predetermined condition as a first main sample on the basis of the past event occurrence data 143 accepted as the input in step S101. In this example, the predetermined condition indicates, for example, a condition such as a period of time between 10:00 and 11:00 on weekdays in September as described above.

In step S103, the CPU 11 outputs event occurrence data that may occur under a condition similar to the above predetermined condition as a first auxiliary sample on the basis of the past event occurrence data 143 accepted as the input in step S101. In this example, the similar condition indicates, for example, a condition such as a period of time between 10:00 and 11:00 on weekdays in August and October before and after September as described above.

In step S104, for example, the CPU 11 creates a plurality of allocation plans of ambulances for a sick/injured person occurrence event on the basis of the above ambulance data 141 in FIG. 7 and the above fire station data 142 in FIG. 8.

In step S105, the CPU 11 applies the first main sample output in step S102 and the first auxiliary sample output in step S103 to each of the plurality of allocation plans created in step S104 and evaluates whether or not each of the plurality of allocation plans satisfies the evaluation criteria. Then, among the plurality of allocation plans, the CPU 11 stores effective allocation plans that satisfy the evaluation criteria in the storage 14 as, for example, the above effective allocation plans 144 in FIG. 11, and the processing in phase 1 by the allocation search program ends.

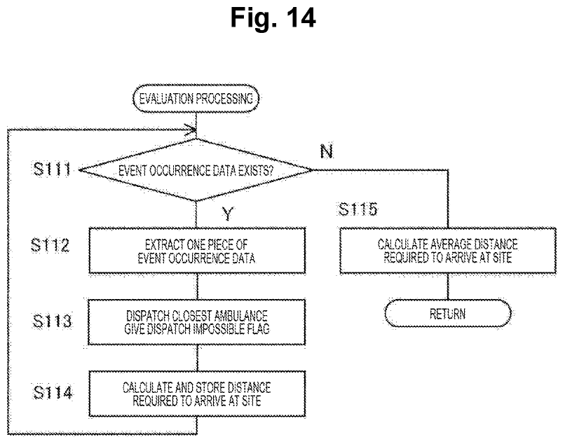

FIG. 14 is a flowchart showing an example of a flow of evaluation processing according to this embodiment. The flow of FIG. 14 specifically shows the evaluation processing in step S105 of FIG. 13.

In step S111 of FIG. 14, the CPU 11 determines whether or not event occurrence data indicating the first main sample or the first auxiliary sample exists. When it is determined that the event occurrence data exists (in a case of positive determination), the processing proceeds to step S112, and, when it is determined that the event occurrence data does not exist (in a case of negative determination), the processing proceeds to step S115.

In step S112, the CPU 11 extracts one piece of the event occurrence data.

In step S113, the CPU 11 dispatches the closest ambulance for the event occurrence data extracted in step S112 and gives, to the dispatched ambulance, a dispatch impossible flag indicating that the dispatched ambulance cannot be allowed for the next dispatch for a certain period of time.

In step S114, the CPU 11 calculates a distance from a fire station where the ambulance to which the dispatch impossible flag is given in step S113 is allocated to a site at which the ambulance arrives, stores the calculated distance in the storage 14, returns to step S111, and repeats the processing for all pieces of the event occurrence data.

Meanwhile, in step S115, the CPU 11 calculates an average distance required to arrive at the site, and returns to step S105 in FIG. 13. As an evaluation method, as described above, an average of distances that dispatchable ambulances existing closest to a site of occurrence of a sick/injured person travel until the ambulances arrive at the site is calculated, and the calculated average travel distance is compared with, for example, an average travel distance in the above original ambulance allocation in FIG. 10.

Next, processing in phase 2 will be described. The processing in phase 2 is executed, for example, at a fixed time before 10:00 (e.g. 9:00) on weekdays in September. The second sample output unit 104 obtains, for example, an occurrence frequency of a sick/injured person in each area between 10:00 and 11:00 on weekdays in the last month on the basis of the latest event occurrence data 145 and samples future pseudo occurrence data on the basis of a result thereof. The latest event occurrence data 145 indicates the latest past sick/injured person occurrence data accumulated by immediately before. The number of days to be sampled is, for example, 100 days. In addition, as in the processing in phase 1 described above, the second main sample output unit 104A and the second auxiliary sample output unit 104B may share roles, increase or decrease the occurrence frequency by a certain value, and output samples, or may generate samples on the basis of the random variable by using MCMC described above in FIG. 6.

The second allocation evaluation unit 105 evaluates all the effective allocation plans 144 accumulated in the storage 14. The evaluation method herein is similar to the evaluation method in the processing in phase 1 described above, except that the acquired allocation plans are not the allocation plans created by the allocation plan creation unit 102, but are the effective allocation plans 144 evaluated by the first allocation evaluation unit 103.

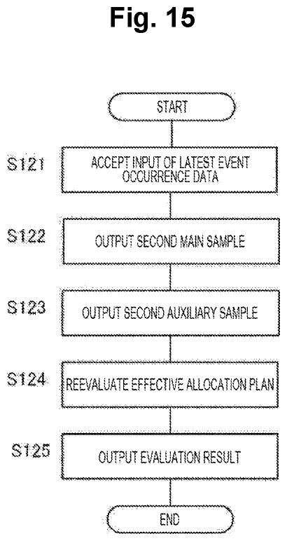

FIG. 15 is a flowchart showing an example of a flow of processing in phase 2 by the allocation search program according to this embodiment. The processing in phase 2 by the allocation search program is implemented by the CPU 11 of the allocation search device 10 writing the allocation search program stored in the ROM 12 or the storage 14 to the RAM 13 and executing the allocation search program.

In step S121 of FIG. 15, the CPU 11 accepts input of the latest event occurrence data 145 indicating, for example, event occurrence data in the last month. In this example, the event occurrence data indicates, for example, the sick/injured person occurrence data as in phase 1 described above.

In step S122, the CPU 11 outputs future event occurrence data that may occur under a predetermined condition as a second main sample on the basis of the latest event occurrence data 145 accepted as the input in step S121. In this example, the predetermined condition indicates, for example, a condition such as a period of time between 10:00 and 11:00 on weekdays in September as in phase 1 described above.

In step S123, the CPU 11 outputs future event occurrence data that may occur under a condition similar to the above predetermined condition as a second auxiliary sample on the basis of the latest event occurrence data 145 accepted as the input in step S121. In this example, the similar condition indicates, for example, a condition such as a period of time between 10:00 and 11:00 on weekdays in August and October before and after September as in phase 1 described above.

In step S124, the CPU 11 applies the second main sample output in step S122 and the second auxiliary sample output in step S123 to each of the effective allocation plans 144 (see FIG. 11) stored in the storage 14 in phase 1 described above and reevaluates whether or not each effective allocation plan 144 satisfies the evaluation criteria. The reevaluation method is similar to the evaluation method in phase 1 described above.

In step S125, the CPU 11 outputs a final evaluation result obtained by the reevaluation in step S124, and the processing in phase 2 by the allocation search program ends.

In this way, the effective allocation plans are evaluated again on the basis of the latest occurrence data rows. As a result, an evaluation result may be different from the above evaluation result in FIG. 11. A user can decide which allocation of resources is finally employed on the basis of the evaluation result.

The processing in phase 2 can be performed by a general-purpose PC in about several tens of seconds to several minutes. Therefore, for example, it is possible to quickly find and employ appropriate allocation of the resources on the basis of the latest event occurrence data on the day when the allocation of the resources is desired to be changed.

The above method can be applied to other cases. For example, in case that the number of ambulance squads is reduced for some reason, it is also possible to obtain effective allocation with a small number of ambulance squads in advance and use the allocation.

As described above, according to this embodiment, in a case where it is necessary to determine effective allocation of resources in a relatively short time, it is possible to obtain robust allocation of the resources that can expect an effect even if a prediction result of occurrence of an event is not completely right.

Allocation search processing that is executed by the CPU reading software (program) in the above embodiment may be executed by various processors other than the CPU. Examples of the processors in this case include a programmable logic device (PLD) whose circuit configuration can be changed after manufacturing, such as a field-programmable gate array (FPGA), and a dedicated electric circuit that is a processor having a circuit configuration exclusively designed for executing specific processing, such as an application specific integrated circuit (ASIC). Further, the allocation search processing may be executed by one of the various processors or may be executed by a combination of two or more processors of the same type or different types (e.g. a combination of a plurality of FPGAs or a combination of a CPU and an FPGA). Furthermore, a hardware structure of the various processors is, more specifically, an electric circuit in which circuit elements such as semiconductor elements are combined.

In the above embodiment, the aspect in which the allocation search program is stored (installed) in advance in the storage has been described, but the embodiment is not limited thereto. The program may be provided by being stored in a non-transitory storage medium such as a compact disk read only memory (CD-ROM), a digital versatile disk read only memory (DVD-ROM), and a universal serial bus (USB) memory. The program may be downloaded from an external device via a network.

Regarding the above embodiment, the following supplementary notes are further disclosed.

### SUPPLEMENTARY NOTE 1

An allocation search device comprising:
a memory; and
at least one processor connected to the memory, wherein:
the processor
outputs, on the basis of event occurrence data obtained in the past, event occurrence data that may occur under a predetermined condition as a first main sample and event occurrence data that may occur under a condition similar to the predetermined condition as a first auxiliary sample;
creates a plurality of allocation plans of resources for an occurring event;
evaluates whether or not each of the plurality of allocation plans satisfies a predetermined evaluation criterion in a case where the first main sample and the first auxiliary sample are applied to each of the plurality of allocation plans;
outputs, on the basis of latest event occurrence data, future event occurrence data that may occur under the predetermined condition as a second main sample and future event occurrence data that may occur under the similar condition as a second auxiliary sample; and
in a case where the second main sample and the second auxiliary sample are applied to each of the allocation plans that satisfy the predetermined evaluation criterion, reevaluates whether or not each of the allocation plans that satisfy the predetermined evaluation criterion satisfies the predetermined evaluation criterion.

### SUPPLEMENTARY NOTE 2

A non-transitory storage medium storing a program executable by a computer to execute allocation search processing,
the allocation search processing comprising:
outputting, on the basis of event occurrence data obtained in the past, event occurrence data that may occur under a predetermined condition as a first main sample and event occurrence data that may occur under a condition similar to the predetermined condition as a first auxiliary sample;
creating a plurality of allocation plans of resources for an occurring event;
evaluating whether or not each of the plurality of allocation plans satisfies a predetermined evaluation criterion in a case where the first main sample and the first auxiliary sample are applied to each of the plurality of allocation plans;
outputting, on the basis of latest event occurrence data, future event occurrence data that may occur under the predetermined condition as a second main sample and future event occurrence data that may occur under the similar condition as a second auxiliary sample; and
in a case where the second main sample and the second auxiliary sample are applied to each of the allocation plans that satisfy the predetermined evaluation criterion, reevaluating whether or not each of the allocation plans that satisfy the predetermined evaluation criterion satisfies the predetermined evaluation criterion.

### REFERENCE SIGNS LIST

10 Allocation search device
11 CPU
12 ROM
13 RAM
14 Storage
15 Input unit
16 Display unit
17 Communication I/F
18 Bus
101 First sample output unit
101A First main sample output unit
101B First auxiliary sample output unit
102 Allocation plan creation unit
103 First allocation evaluation unit
104 Second sample output unit
104A Second main sample output unit
104B Second auxiliary sample output unit
105 Second allocation evaluation unit
106 Result output unit
141 Ambulance data
142 Fire station data
143 Past event occurrence data
144 Effective allocation plan
145 Latest event occurrence data

1. An allocation search device comprising a processor configured to execute an operation comprising:
generating, based on a plurality of event occurrence data, first event occurrence data and second event occurrence data, wherein the first event occurrence data are associated with a predetermined condition as a first main sample, and the second event occurrence data are associated with a condition that is similar to the predetermined condition as a first auxiliary sample;
creating a plurality of allocation plans of resources associated with an event;
identifying a first set of allocation plan of the plurality of allocation plans based on determining whether or not each of the plurality of allocation plans satisfies a predetermined evaluation criterion, wherein the predetermined evaluation criterion includes a case where the first main sample and the first auxiliary sample are applied to each of the plurality of allocation plans;
generating, based on the latest event occurrence data associated with the most recently occurred event, first future event occurrence data and second future event occurrence data, wherein the first future event occurrence data are associated with the predetermined condition as a second main sample, and wherein the second future event occurrence data are associated with the similar condition as a second auxiliary sample; and
updating the first set of allocation plans of the plurality of allocation plans based on determining whether or not each of the allocation plans satisfies the predetermined evaluation criterion when the second main sample and the second auxiliary sample are applied to each of the plurality of allocation.

2. The allocation search device according to claim 1, wherein the predetermined evaluation criterion includes:
a first subset main criterion associated with the first main sample
a first subset auxiliary criterion associated with the first auxiliary sample,
a second subset main criterion associated with the second main sample,
a second subset auxiliary criterion associated with the second auxiliary sample,
the first subset main criterion is distinct from the first subset auxiliary criterion, and
the second subset main criterion is distinct from the second subset auxiliary criterion.

3. The allocation search device according to claim 1, wherein
the generating the first event occurrence data and the second event occurrence data further comprises:
outputting the first main sample; and
outputting the first auxiliary sample; and wherein
the generating the first future event occurrence data and the second future event occurrence data further comprises:
outputting the second main sample; and
outputting the second auxiliary sample.

4. The allocation search device according to claim 1, wherein
the first main sample indicates a first pseudo occurrence data series generated according to simulation based on a first event occurrence frequency, the first event occurrence frequency is obtained in each predetermined area associated with a first data series that depicts actual occurrences under the predetermined condition, and
the first auxiliary sample indicates a second pseudo occurrence data series generated according to simulation based on a second event occurrence frequency, and the second event occurrence frequency is obtained in each predetermined area associated with a second data series that depicts actual occurrences under the similar condition.

5. The allocation search device according to claim 1, wherein
the first main sample indicates a first pseudo occurrence data series generated according to simulation based on a variable of a first event occurrence probability, the event occurrence probability is obtained on the basis of a data series that actually occurs in the predetermined condition, and wherein
the first auxiliary sample indicates a second pseudo occurrence data series generated according to simulation based on a variable of a second event occurrence probability, and the second event occurrence probability is obtained on the basis of a data series that actually occurs under the similar condition.

6. An allocation search method comprising:
generating, based on a plurality of event occurrence data, first event occurrence data and second event occurrence data, wherein the first event occurrence data are associated with a predetermined condition as a first main sample, and the second event occurrence data are associated with a condition that is similar to the predetermined condition as a first auxiliary sample;
creating a plurality of allocation plans of resources associated with an event;
identifying a first set of allocation plan of the plurality of allocation plans based on determining whether or not each of the plurality of allocation plans satisfies a predetermined evaluation criterion, wherein the predetermined evaluation criterion includes a case where the first main sample and the first auxiliary sample are applied to each of the plurality of allocation plans;
generating, based on the latest event occurrence data associated with the most recently occurred event, first future event occurrence data second future event occurrence data, wherein the first future occurrence data are associated with the predetermined condition as a second main sample, and wherein the second future event occurrence data are associated with the similar condition as a second auxiliary sample; and
updating the first set of allocation plans of the plurality of allocation plans based determining whether or not each of the allocation plans satisfies the predetermined evaluation criterion when the second main sample and the second auxiliary sample are applied to each of the plurality of allocation plans.

7. A computer-readable non-transitory recording medium storing computer-executable program instructions that when executed by a processor cause a computer system to execute an operation comprising:
generating, based on a plurality of event occurrence data, first event occurrence data and second event occurrence data, wherein the first event occurrence data are associated with a predetermined condition as a first main sample, and the second event occurrence data are associated with a condition that is similar to the predetermined condition as a first auxiliary sample;
creating a plurality of allocation plans of resources associated with an event;
identifying a first set of allocation plan of the plurality of allocation plans based on determining whether or not each of the plurality of allocation plans satisfies a predetermined evaluation criterion, wherein the predetermined evaluation criterion includes a case where the first main sample and the first auxiliary sample are applied to each of the plurality of allocation plans;
generating, based on the latest event occurrence data associated with the most recently occurred event, first future event occurrence data and second future event occurrence data, wherein the first future event occurrence data are associated with the predetermined condition as a second main sample, and wherein the second future event occurrence data are associated with the similar condition as a second auxiliary sample; and
updating the first set of allocation plans of the plurality of allocation plans based on determining whether or not each of the allocation plans satisfies the predetermined evaluation criterion when the second main sample and the second auxiliary sample are applied to each of the plurality of allocation plans.

8. The allocation search device according to claim 2, wherein:
the generating the first event occurrence data and the second event occurrence data further comprises:
outputting the first main sample; and
outputting the first auxiliary sample; and
the generating the first future event occurrence data and the second future event occurrence data further comprises:
outputting the second main sample; and
outputting the second auxiliary sample.

9. The allocation search device according to claim 2, wherein:
the first main sample indicates a first pseudo occurrence data series generated according to simulation based on a first event occurrence frequency, the first event occurrence frequency is obtained in each predetermined area associated with a first data series that depicts actual occurrences under the predetermined condition, and wherein
the first auxiliary sample indicates a second pseudo occurrence data series generated according to simulation based on a second event occurrence frequency, and the second event occurrence frequency is obtained in each predetermined area associated with a second data series that depicts actual occurrences under the similar condition.

10. The allocation search device according to claim 2, wherein:
the first main sample indicates a first pseudo occurrence data series generated according to simulation based on a variable of a first event occurrence probability, the event occurrence probability is obtained on the basis of a data series that actually occurs in the predetermined condition, and wherein
the first auxiliary sample indicates a second pseudo occurrence data series generated according to simulation based on a variable of a second event occurrence probability, and the second event occurrence probability is obtained on the basis of a data series that actually occurs under the similar condition.

11. The allocation search method according to claim 6, wherein
the predetermined evaluation criterion includes:
a first subset main criterion associated with the first main sample
a first subset auxiliary criterion associated with the first auxiliary sample,
a second subset main criterion associated with the second main sample,
a second subset auxiliary criterion associated with the second auxiliary sample,
the first subset main criterion is distinct from the first subset auxiliary criterion, and
the second subset main criterion is distinct from the second subset auxiliary criterion.

12. The allocation search method according to claim 6, the generating the first event occurrence data and the second event occurrence data further comprises:
outputting the first main sample; and
outputting the first auxiliary sample; and
the generating the first future event occurrence data and the second future event occurrence data further comprises:
outputting the second main sample; and
outputting the second auxiliary sample.

13. The allocation search method according to claim 6, wherein
the first main sample indicates a first pseudo occurrence data series generated according to simulation based on a first event occurrence frequency, the first event occurrence frequency is obtained in each predetermined area associated with a first data series that depicts actual occurrences under the predetermined condition, and wherein
the first auxiliary sample indicates a second pseudo occurrence data series generated according to simulation based on a second event occurrence frequency, and the second event occurrence frequency is obtained in each predetermined area associated with a second data series that depicts actual occurrences under the similar condition.

14. The allocation search method according to claim 6, wherein
the first main sample indicates a first pseudo occurrence data series generated according to simulation based on a variable of a first event occurrence probability, the event occurrence probability is obtained on the basis of a data series that actually occurs in the predetermined condition, and wherein
the first auxiliary sample indicates a second pseudo occurrence data series generated according to simulation based on a variable of a second event occurrence probability, and the second event occurrence probability is obtained on the basis of a data series that actually occurs under the similar condition.

15. The allocation search method according to claim 11, the generating the first event occurrence data and the second event occurrence data further comprises:
outputting the first main sample; and
outputting the first auxiliary sample; and
the generating the first future event occurrence data and the second future event occurrence data further comprises:
outputting the second main sample; and
outputting the second auxiliary sample.

16. The allocation search method according to claim 11, the generating the first event occurrence data and the second event occurrence data further comprises:
outputting the first main sample; and
outputting the first auxiliary sample; and
the generating the first future event occurrence data and the second future event occurrence data further comprises:
outputting the second main sample; and
outputting the second auxiliary sample.

17. The allocation search method according to claim 11, wherein
the first main sample indicates a first pseudo occurrence data series generated according to simulation based on a first event occurrence frequency, the first event occurrence frequency is obtained in each predetermined area associated with a first data series that depicts actual occurrences under the predetermined condition, and wherein
the first auxiliary sample indicates a second pseudo occurrence data series generated according to simulation based on a second event occurrence frequency, and the second event occurrence frequency is obtained in each predetermined area associated with a second data series that depicts actual occurrences under the similar condition.

18. The allocation search method according to claim 11, wherein
the first main sample indicates a first pseudo occurrence data series generated according to simulation based on a variable of a first event occurrence probability, the event occurrence probability is obtained on the basis of a data series that actually occurs in the predetermined condition, and wherein
the first auxiliary sample indicates a second pseudo occurrence data series generated according to simulation based on a variable of a second event occurrence probability, and the second event occurrence probability is obtained on the basis of a data series that actually occurs under the similar condition.

19. The computer-readable non-transitory recording medium according to claim 7, wherein
the predetermined evaluation criterion includes:
a first subset main criterion associated with the first main sample
a first subset auxiliary criterion associated with the first auxiliary sample,
a second subset main criterion associated with the second main sample,
a second subset auxiliary criterion associated with the second auxiliary sample,
the first subset main criterion is distinct from the first subset auxiliary criterion, and
the second subset main criterion is distinct from the second subset auxiliary criterion.

20. The computer-readable non-transitory recording medium according to claim 7, wherein
the first main sample indicates a first pseudo occurrence data series generated according to simulation based on a first event occurrence frequency, the first event occurrence frequency is obtained in each predetermined area associated with a first data series that depicts actual occurrences under the predetermined condition, and wherein
the first auxiliary sample indicates a second pseudo occurrence data series generated according to simulation based on a second event occurrence frequency, and the second event occurrence frequency is obtained in each predetermined area associated with a second data series that depicts actual occurrences under the similar condition.
